(19) United States
(12) Patent Application Publication
(10) Pub. No.: US 2012/0041071 A1
(43) Pub. Date: Feb. 16, 2012

(54) TAPENTADOL FOR TREATING PAIN DUE TO OSTEOARTHRITIS

(75) Inventors: Ferdinand ROMBOUT, NL-Klimmen (NL); Claudia LANGE, Duesseldorf (DE)

(73) Assignee: Gruenenthal GmbH, Aachen (DE)

(21) Appl. No.: 13/279,663

(22) Filed: Oct. 24, 2011

Related U.S. Application Data
(63) Continuation of application No. 12/875,728, filed on Sep. 3, 2010, now abandoned.
Application No. 12/875,728 is a continuation of application No. 12/106,626, filed on Apr. 21, 2008, now abandoned.
(60) Provisional application No. 60/907,940, filed on Apr. 23, 2007.

(30) Foreign Application Priority Data
Apr. 23, 2007 (DE) DE 102007019417.1

(51) Int. Cl. A61K 31/137; A61P 25/00

(52) U.S. Cl. 514/654

(57) ABSTRACT

The use of tapentadol for treating pain due to osteoarthritis.

### CROSS REFERENCE TO RELATED APPLICATIONS

This application is a continuation of application Ser. No. 12/875,728, filed Sep. 3, 2010, which was a continuation of application Ser. No. 12/106,626, filed Apr. 21, 2008 and now abandoned, which, in turn, claimed priority from U.S. provisional patent application No. 60/907,940, filed Apr. 23, 2007.

### BACKGROUND OF THE INVENTION

The invention relates to the use of tapentadol for treating pain due to osteoarthritis.

Arthrosis (osteoarthritis, arthrosis deformans) is the most widespread human joint disease. It is a dynamic, but slow progressing, degenerative disease of the cartilage and other articular tissue, particularly in elderly individuals, with intermittent inflammatory episodes. It may be distinguished from other rheumatic diseases by the absence of inflammatory parameters, restricted mobility, short-term articular stiffness and its radiological features.

Arthrosis or joint wear and tear is joint damage that starts with the degradation of the articular cartilage. In severe cases, it finally results in transformation processes in the adjacent bone and the surface of the joint is destroyed. Therefore, the consequences of the disease are pain and stiffness of the joint with restricted movement. The joints can become deformed and are ultimately completely ossified. Arthrosis generally progresses slowly. As a result, the layer of cartilage becomes thicker at first and the chondrocytes become more metabolically active. Changes to the subchondral trabecula result in reduced pressure relief by the spongy bone. The reparation tissue is subjected to more stress and as the duration of the disease advances, the equilibrium alters with respect to destruction. X-rays reveal a narrowing of the articular space and osteophytes form at the edges. For further details, it is possible, for example, to refer comprehensively to D Höffler et al, AVP Therapieempfehlungen der Arzneimittelkommission der Deutschen Ärzteschaft, Arzneiverordnung in der Praxis, “Degenerative Gelenkerkrankungen”, 2nd Edition 2001; and H Bröll et al, CliniCum, Special Edition September 2001, Konsensus-Statement, “Arthrose-Diagnostik % Therapie”.

In principle, all joints can be affected by arthrotic changes. However, most commonly affected are the knee joints (gonarthrosis) and hip joints (coxarthrosis) which have to bear a great amount of weight. The disease also frequently occurs in the small vertebral joints (spondylarthrosis) and in the finger joints. ICD-10 defines arthrosis of the hips and knees as primary cartilage diseases associated with painful restrictions of movement (pain following periods of inactivity, weight-bearing pain) or difficulty in walking. Inflammation, such as synovitis, can, but does not have to become established.

Cardinal and early symptoms of arthrosis are pain (early triad: pain following periods of inactivity, fatigue-induced pain, weight-bearing pain; late triad: constant pain, night pain, muscular pain). These are accompanied by restrictions on movement, sensitivity to changes in the weather and crepitation. The causes of pain with arthrosis are primarily the result of irritation in the periarticular tendon and ligament attachments, secondary inflammation, distension of the joint capsule, reactive effusion, increased pressure in the subchondral bone and microfractures.

In early stages, pain only occurs on weight-bearing and subsides if movement is continued, eg walking further, after a few minutes. When accompanied by inflammation, these are the typical symptoms of activated arthrosis: the joint is painful, feels warm and is swollen. Mobility is restricted. The inflammation often subsides even without treatment. This explains the generally episodic course of arthrosis: phases of more severe pain and restricted movement alternate with phases of less pain and good mobility. The more advanced the signs of wear and tear, the more rapidly one pain phase succeeds another. Ultimately, the pain is constant.

There are a variety of drug-based and non-drug based treatments available which may be used individually or in combination:
general measures, eg swimming, targeted gymnastics, use of walking aids, diet, etc.;
physical therapies, eg heat packs, electrotherapy and kinesiotherapy, etc.;
pharmacotherapy;
orthopaedic techniques, eg bandages, ortheses, etc; and
operative therapy, eg transplantation of autologous cartilage cells, artificial joint replacement, etc.

The European League Against Rheumatism (EULAR) recommends that the Lequesne Index, ie an overall evaluation by the doctor and the patient's assessment of the pain, be used to assess the success of a specific therapy. In addition to an assessment of swelling, reddening and resistance to pressure of the joint, the FDA recommend that the pain and function be assessed by means of the Western-Ontario-McMaster-Universities-Osteoarthritis-Index (WOMAC) and the Lequesne Index. For drugs used for the symptomatic treatment of arthrosis, the Osteoarthritis Research Society recommends the scales for the WOMAC pain score as the main target criterion and the WOMAC mobility restriction score or Lequesne Index as the secondary target criterion plus an overall assessment by the doctor and patients.

The pharmacotherapeutic spectrum of the groups of active substances available to treat arthrosis includes
non-opioid analgesics, eg paracetamol;
nonsteroidal anti-inflammatory drugs (NSAIDs), eg acemetacin, acetylsalicylic acid, aceclofenac, diclofenac, ibuprofen, ketoprofen, mefenamic acid; tiaprofenic acid, indometacin, lonazolac, naproxen, proglumetacin, meloxicam, piroxicam, rofecoxib, celecoxib;
opioid analgesics, eg dihydrocodeine, tramadol, tilidine-naloxone, morphine, buprenorphine, oxycodone, fentanyl and hydromorphone;
percutaneously administered antiphlogistics and hyperaemic agents;
glucocorticosteroid crystal suspensions for intraarticular injections; and
other active substances for oral or intraarticular injections, eg glucosamine, ademetionine, oxaceprol, hyaluronic acid, etc.

Opioid analgesics do not belong to the routine repertoire of drug treatment for arthrosis, but are unavoidable in certain situations. However, conventional opioid analgesics sometimes have significant side effects, in particular constipation, nausea, vomiting, headache, sedation, fatigue, respiratory depression, allergies and sometimes a drop in blood pressure. These side effects complicate the long-term therapy of chronic pain with arthrosis. Therefore, treatment with conventional opioid analgesics is generally indicated when all other therapeutic options have been exhausted, for example in the case of patients who cannot undergo an operation but are suffering extreme pain at rest which fails to respond to other substances with an analgesic action.

There is a need for alternative pharmacotherapeutic methods for arthrosis characterised by effective pain control and a reduced side-effects profile.

### SUMMARY OF THE INVENTION

Therefore, it was the object of the invention to find compounds that are effective in pain control with arthrosis and have advantages over conventional analgesics.

These and other objects have been achieved by the invention as described and claimed hereinafter.

The invention relates to the use of tapentadol to produce a medicine for treating pain due to arthrosis. It has surprisingly been found that tapentadol, preferably as a prolonged release (PR) formulation (synonym of extended release (ER) formulation), ie a formulation with extended release within the meaning of the European Pharmacopoeia, combines excellent efficacy for the treatment of pain due to arthrosis with a reduced side effect spectrum. Extended release is usually understood to mean modified release which differs from the release of conventional pharmaceutical forms administered via the same route. The modification of the release is usually achieved by a special design of the pharmaceutical form or a special production method.

### BRIEF DESCRIPTION OF THE DRAWINGS

FIG. 1 shows a schematic representation of the titration scheme adhered to during the investigation of the efficacy of tapentadol for treating pain due to arthrosis.

FIG. 2 shows a schematic representation of the efficacy of tapentadol (100 mg and 200 mg) compared to a placebo and oxycodone.

FIG. 3 shows a mathematical evaluation of the distribution of the serum concentration within a patient population following the administration of different doses of tapentadol.

FIG. 4 shows a mathematical evaluation of the connection between the serum concentration of tapentadol and its effect with respect to pain alleviation in a patient population on the basis of data from various clinical studies.

### DETAILED DESCRIPTION OF THE INVENTION

Tapentadol, ie (−)-(1R,2R)-3-(3-dimethylamino-1-ethyl-2-methyl-propyl)-phenol, is a synthetic, centrally acting analgesic which is effective in the treatment of moderate to severe, acute or chronic pain.

Tapentadol exhibits a dual mechanism of action, on the one hand as a μ-opioid receptor agonist and on the other as a noradrenaline transporter inhibitor. In humans, the affinity of tapentadol to the recombinantly produced μ-opioid receptor is 18-times less than that of morphine. However, clinical studies have shown the pain-alleviating action of tapentadol to be only two to three times less than that of morphine. The only slightly reduced analgesic efficacy with a simultaneously 18-times reduced affinity to the recombinant μ-opioid receptor indicates that the noradrenaline transporter inhibiting property of tapentadol also contributes to its analgesic efficacy. Consequently, it may be assumed that tapentadol has a similar analgesic efficacy to that of pure μ-opioid receptor agonists but has fewer of the side effects associated with the μ-opioid receptor. The compound can be used in the form of its free base or as a salt or solvate. The production of the free base is known for example from EP-A 693 475.

For the purposes of the description, “tapentadol” means (−)-(1R,2R)-3-(3-dimethylamino-1-ethyl-2-methyl-propyl)-phenol and the pharmaceutically acceptable salts and solvates thereof.

Suitable pharmaceutically acceptable salts include salts of inorganic acids, such as eg hydrogen chloride, hydrogen bromide and sulfuric acid, and salts of organic acids, such as methanesulfonic acid, fumaric acid, maleic acid, acetic acid, oxalic acid, succinic acid, malic acid, tartaric acid, mandelic acid, lactic acid, citric acid, glutaminic acid, acetylsalicylic acid, nicotinic acid, aminobenzoic acid, α-lipoic acid, hippuric acid and aspartic acid. The preferred salt is hydrochloride.

In one preferred embodiment, the medicine is a solid medicinal form. Preferably, the medicine is formulated for oral administration. However, other pharmaceutical forms are also possible for example buccal, sublingual, transmucosal, rectal, intralumbar, intraperitoneal, transdermal, intravenous, intramuscular, intragluteal, intracutaneous and subcutaneous.

Depending upon the formulation, the medicine preferably contains suitable additives and/or excipients. Suitable additives and/or excipients for the purpose of the invention are all substances for achieving galenic formulations known to the person skilled in the art from the prior art. The selection of these excipients and the amounts to use depend upon how the medicinal product is to be administered, ie orally, intravenously, intraperitoneally, intradermally, intramusuclarly, intranasally, buccally or topically.

Suitable orally administrable preparation forms include tablets, chewable tablets, dragees, capsules, granules, drops, juices or syrups; suitable for parenteral, topical and inhalative administration are solutions, suspensions, easily reconstituted dry preparations and sprays. A further possibility are suppositories for use in the rectum. Use in a depot in dissolved form, a carrier foil or a plaster, optionally with the addition of means to encourage penetration of the skin, are examples of suitable percutaneous administration forms.

Examples of suitable excipients and additives for oral administration forms include disintegrants, lubricants, binders, fillers, mould release agents, optionally solvents, flavourings, sugar, in particular carriers, diluents, colorants, antioxidants, etc.

For suppositories, it is possible to use inter alia waxes or fatty acid esters and for parenteral means of application, carriers, preservatives, suspension aids, etc.

Excipients can be for example: water, ethanol, 2-propanol, glycerin, ethylene-glycol, propylene glycol, polyethylene glycol, polypropylene glycol, glucose, fructose, lactose, sucrose, dextrose, molasses, starch, modified starch, gelatin, sorbitol, inositol, mannitol, microcrystalline cellulose, methyl cellulose, carboxymethylcellulose, cellulose acetate, shellac, cetyl alcohol, polyvinylpyrrolidone, paraffins, waxes, natural and synthetic rubbers, acacia gum, alginates, dextran, saturated and unsaturated fatty acids, stearic acid, magnesium stearate, zinc stearate, glyceryl stearate, sodium lauryl sulfate, edible oils, sesame oil, coconut oil, groundnut oil, soybean oil, lecithin, sodium lactate, polyoxyethylene and propylene fatty acid ester, sorbitan fatty acid esters, sorbic acid, benzoic acid, citric acid, ascorbic acid, tannic acid, sodium chloride, potassium chloride, magnesium chloride, calcium chloride, magnesium oxide, zinc oxide, silicon dioxide, titanium oxide, titanium dioxide, magnesium sulfate, zinc sulfate, calcium sulfate, potash, calcium phosphate, dicalcium phosphate, potassium bromide, potassium iodide, talc kaolin, pectin, crospovidone, agar and bentonite.

The medicinal product and pharmaceutical compositions is performed with the aid of means, devices, methods and processes which are well known in the prior art of pharmaceutical formulation, such as those described for example in “Remington's Pharmaceutical Sciences”, ed AR Gennaro, 17th edition, Mack Publishing Company, Easton, pa. (1985), in particular in Part 8, Chapters 76 to 93.

For example, for a solid formulation, such as a tablet, the active substance of the medicinal product can be granulated with a pharmaceutical carrier, eg conventional tablet ingredients, such as maize starch, lactose, sucrose, sorbitol, talc, magnesium stearate, dicalcium phosphate or pharmaceutically acceptable rubbers, and pharmaceutical diluents, such as water, for example, to form a solid composition containing the active substance in a homogeneous distribution. Here, a homogeneous distribution should be understood as meaning that the active substance is distributed uniformly throughout the entire composition so that this can be easily divided into equally effective single dose forms, such as tablets, capsules, dragees. The solid composition is then divided into single dose forms. The tablets or pills can also be coated or compounded in some other way in order to produce a dosage form with delayed release. Suitable coating means are inter alia polymers acids and mixtures of polymeric acids with materials such as shellac, for example, cetyl alcohol and/or cellulose acetate.

The amounts of tapentadol to be administered to patients vary depending upon the weight of the patient, the method of administration and the severity of the disease. In a preferred embodiment, the medicine contains tapentadol in a amount of 10 to 300 mg, more preferably 20 to 290 mg, even more preferably 30 to 280 mg, most preferably 40 to 260 mg, as an equivalent dose based on the free base.

Delayed release of tapentadol is possible from formulations for oral, rectal or percutaneous administration. Preferably, the medicine is formulated for once-daily administration, for twice-daily administration (bid) or for thrice-daily administration, with twice-daily administration (bid) being particularly preferred.

The delayed release of tapentadol can, for example, be achieved by retardation by means of a matrix, a coating or release systems with an osmotic action (see eg US-A-2005-58706).

In one preferred embodiment, the mean serum concentration of tapentadol, following twice-daily administration of the medicine over a period of at least three days, more preferably at least four days and in particular at least five days, is on average at least 5.0 ng/ml, at least 10 ng/ml, at least 15 ng/ml or at least 20 ng/ml, more preferably at least 25 ng/ml or at least 30 ng/ml, even more preferably at least 35 ng/ml or at least 40 ng/ml, most preferably at least 45 ng/ml or at least 50 ng/ml and in particular at least 55 ng/ml or at least 60 ng/ml. This means that tapentadol is administered over a period of at least three days twice daily and then, preferably 2 h after the administration, the serum concentration is measured. The authoritative numerical value is then obtained as the mean value for all the patients investigated.

In another preferred embodiment, the mean serum concentration of tapentadol in at the most 50% of the patient population, which preferably comprises at least 100 patients, more preferably in at the most 40%, even more preferably in at the most 30%, most preferably in at the most 20% and in particular in at the most 10% of the patient population, following twice-daily administration over a period of at least three days, more preferably at least four days and in particular at least five days, is on average less than 5.0 ng/ml, preferably less than 7.5 ng/ml, even more preferably less than 10 ng/ml, most preferably less than 15 ng/ml and in particular less than 20 ng/ml.

In another preferred embodiment, the mean serum concentration of tapentadol in at the most 50% of the patient population, comprising preferably at least 100 patients, more preferably in at the most 40%, even more preferably in at the most 30%, most preferably in at the most 20% and in particular in at the most 10% of the patient population, following twice-daily administration over a period of at least three days, more preferably at least four days and in particular at least five days, is on average more than 300 ng/ml, more preferably more than 275 ng/ml, even more preferably more than 250 ng/ml, most preferably more than 225 ng/ml and in particular more than 200 ng/ml.

Preferably, the mean serum concentration of tapentadol in at least 50% or 55% of the patient population, which preferably comprises at least 100 patients, more preferably in at least 60% or 65%, even more preferably in at least 70% or 75%, most preferably in at least 80% or 85% and in particular in at least 90% or 95% of the patient population, following twice-daily administration over a period of at least three days, more preferably at least four days and in particular at least five days, is on average in the range of from 1.0 ng/ml to 500 ng/ml, more preferably in the range of from 2.0 ng/ml to 450 ng/ml, even more preferably in the range of from 3.0 ng/ml to 400 ng/ml, most preferably in the range of from 4.0 ng/ml to 350 ng/ml and in particular in the range of from 5.0 ng/ml to 300 ng/ml.

In another preferred embodiment, the percentage standard deviation (coefficient of variation) of the mean serum concentration of tapentadol, preferably in a patient population of 100 patients, following twice-daily administration of the medicine over a period of at least three days, more preferably at least four days and in particular at least five days, is at the most ±90%, more preferably at the most ±70%, even more preferably at the most ±50%, at the most ±45% or at the most ±40%, most preferably at the most ±35%, at the most ±30% or at the most ±25% and in particular at the most ±20%, at the most ±15% or at the most ±10%.

Preferably, the serum concentrations are average values, produced from measurements on a patient population of preferably at least 10, more preferably at least 25, even more preferably at least 50, even more preferably at least 75, most preferably at least 100 and in particular at least 250 patients. A person skilled in the art knows how to determine the serum concentrations of tapentadol. In this context, reference is made, for example, to T M Tschentke et al, Drugs of the Future, 2006, 31(12), 1053.

In a preferred embodiment:
the medicine is formulated for oral administration;
the medicine is a solid and/or pressed and/or film-coated medicinal form; and/or
the medicine tapentadol has delayed release from a matrix; and/or
contains the medicine tapentadol in a amount of 0.001 to 99.999% by weight, more preferably 0.1 to 99.9% by weight, even more preferably 1.0 to 99.0% by weight, even more preferably 2.5 to 80% by weight, most preferably 5.0 to 50% by weight and in particular 7.5 to 40% by weight, based on the total weight of the medicine; and/or
the medicine contains a pharmaceutically acceptable carrier and/or pharmaceutically acceptable excipients; and/or
the medicine has a total mass in the range of from 25 to 2,000 mg, more preferably 50 to 1,800 mg, even more preferably 60 to 1,600 mg, even more preferably 70 to 1,400 mg, most preferably 80 to 1,200 mg and in particular 100 to 1,000 mg, and/or
the medicine is selected from the group consisting of tablets, capsules, pellets and granules.

The medicine can be provided as a simple tablet and as a coated tablet (eg as a film-coated tablet or dragee). The tablets are usually round and biconvex, but oblong shapes are also possible. Granules, spheroids, pellets or microcapsules, which are used to fill sachets or capsules or pressed into disintegrating tablets, are also possible.

Medicines containing at least 0.001 to 99.999% tapentadol, in particular low, active doses, are preferred in order to avoid side effects. The medicine contains preferably 0.01% by weight to 99.99% by weight tapentadol, more preferably 0.1 to 90% by weight, even more preferably 0.5 to 80% by weight, most preferably 1.0 to 50% by weight and in particular 5.0 to 20% by weight. To avoid side effects, it may be advantageous at the start of the treatment to increase the amount of tapentadol to be administered gradually (titration) to allow the body to become accustomed to the active substance slowly. Preferably, tapentadol is first administered in a dose which is below the analgesically active dose.

Particularly preferably, the medicine has an oral pharmaceutical form, which is formulated for twice-daily administration and contains tapentadol in an amount of 20 to 260 mg as an equivalent dose based on the free base.

According to the invention, tapentadol is used for treating pain due to arthrosis. Preferably, the arthrosis is selected from the group consisting of gonarthrosis, coxarthrosis and spondylarthrosis.

Preferably, the painful arthrosis is an arthrosis as defined by ICD-10 (International Statistical Classification of Diseases and Related Health Problems, WHO edition, preferably 2007 version). Preferably, the arthrosis is selected from polyarthrosis [M15], coxarthrosis [M16], gonarthrosis [M17], arthrosis of the first carpometacarpal joint [M18], other arthrosis [M19] and spondylosis [M47]. The references in brackets refer to the ICD-10 nomenclature.

If the arthrosis is polyarthrosis [M15], this is preferably selected from the group consisting of primary, generalised (osteo)arthrosis [M15.0], Heberden's nodes (with arthropathy) [M15.1], Bouchard's nodes (with arthropathy) [M15.2], secondary, multiple arthrosis (post-traumatic polyarthrosis) [M15.3], erosive (osteo)arthrosis [M15.4], other polyarthrosis [M15.8] and unspecified polyarthrosis (generalised (osteo)arthrosis not otherwise specified) [M15.9].

If the arthrosis is coxarthrosis [M16], this is preferably selected from the group consisting of bilateral primary coxarthrosis [M16.0], other primary coxarthrosis (unilateral or not otherwise specified) [M16.1], bilateral coxarthrosis resulting from dysplasia [M16.2], other dysplastic coxarthrosis (unilateral or not otherwise specified) [M16.3], bilateral, post-traumatic coxarthrosis [M16.4], other post-traumatic coxarthrosis [M16.5] (unilateral or not otherwise specified), other, bilateral secondary coxarthrosis [M16.6], other secondary coxarthrosis (unilateral or not otherwise specified) [M16.7] and unspecified coxarthrosis [M16.9].

If the arthrosis is gonarthrosis [M17], this is preferably selected from the group consisting of bilateral primary gonarthrosis [M17.0], other primary gonarthrosis (unilateral or not otherwise specified) [M17.1], bilateral, post-traumatic gonarthrosis [M17.2], other post-traumatic gonarthrosis [M17.3] (unilateral or not otherwise specified), other, bilateral secondary gonarthrosis [M17.4], other secondary gonarthrosis (unilateral or not otherwise specified) [M17.5] and unspecified gonarthrosis [M17.9].

If the arthrosis is arthrosis of the first carpometacarpal joint [M18], this is preferably selected from the group consisting of bilateral primary arthrosis of the first carpometacarpal joint [M18.0], other primary arthrosis of the first carpometacarpal joint (unilateral or not otherwise specified) [M18.1], bilateral, post-traumatic arthrosis of the first carpometacarpal joint [M18.2], other post-traumatic arthrosis of the first carpometacarpal joint [M18.3] (unilateral or not otherwise specified), other, bilateral secondary arthrosis of the first carpometacarpal joint [M18.4], other secondary arthrosis of the first carpometacarpal joint (unilateral or not otherwise specified) [M18.5] and unspecified arthrosis of the first carpometacarpal joint [M18.9].

If the arthrosis is other arthrosis [M19], this is preferably selected from the group consisting of primary arthrosis of other joints (primary arthrosis not otherwise specified) [M19.0], post-traumatic arthrosis of other joints (post-traumatic arthrosis not otherwise specified) [M19.1], other secondary arthrosis (secondary arthrosis not otherwise specified) [M19.2], other specified arthrosis [M19.8] and unspecified arthrosis [M19.9].

Preferably, the pain moderate to strong. In a preferred embodiment, the pain is selected from the group consisting of pain following periods of inactivity, weight-bearing pain, fatigue-induced pain, periarticular pain on pressure, radiating pain (eg knee pain with coxarthrosis), pain at rest after spending a long time in the same position, constant pain, spontaneous pain, pain on movement, night pain, muscular pain, pain at the end of the range of movement, osseous pain as spontaneous pain and pain at rest.

Even if the medicines according to the invention exhibit few side effects only, it may be advantageous, for example, to avoid certain types of dependency also to use morphine antagonists, in particular naloxone, naltrexone and/or levallorphan, in addition to tapentadol.

The invention furthermore relates to a method for treating pain due to arthrosis, in which tapentadol is administered to a patient in a pharmaceutically acceptable amount.

The following examples serve for a further explanation of the invention but should not be construed as restrictive.

### Example 1

### Objective

The efficacy and tolerability of tapentadol with prolonged release (prolonged release (PR)) and oxycodone HCl with controlled release (controlled release (CR)) were compared with a placebo in patients with moderate to severe pain due to arthrosis of the knee.

### Methods (Randomised, Placebo-Controlled Double-Blind Study):

Patients (N=670) were randomly selected and treated over 28 days twice with tapentadol PR 100 mg, with tapentadol PR 200 mg, with oxycodone HCl CR 20 mg or with a placebo. The dose was titrated at the start of the treatment. The primary efficacy endpoint was the average perception of pain during the preceding 24 hours at the time of the last medical examination (final visit) based on a visual analog 100-mm sale (VAS, 0 mm=no pain, 100 mm=worst pain imaginable)).

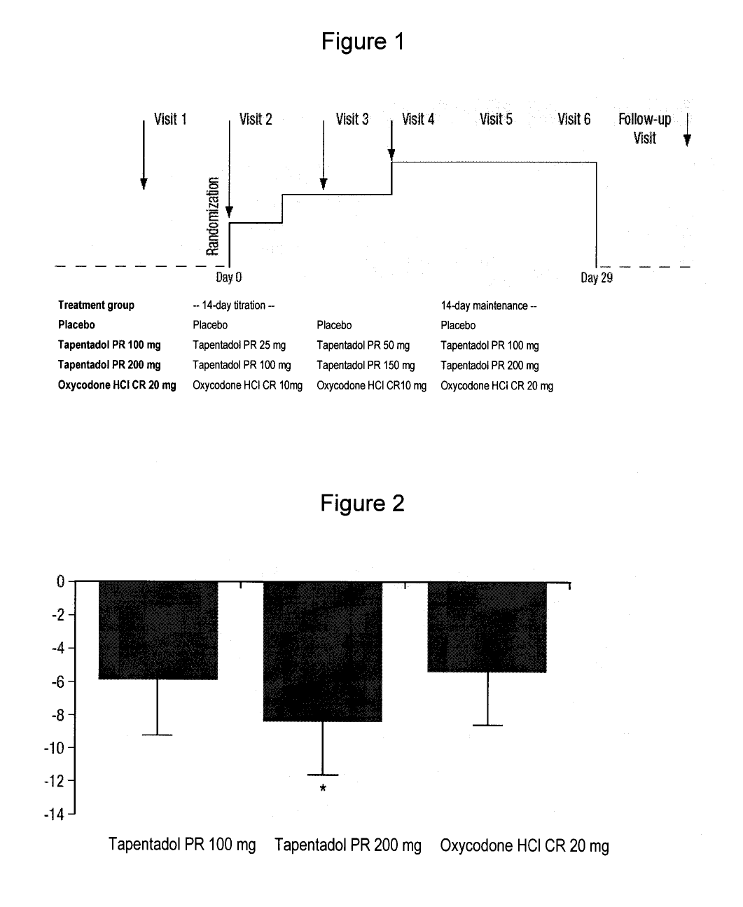

The study consisted of a 14-day, double-blind titration phase (3 days->11 days) followed by a 14-day double-blind maintenance phase (at the highest dose of the titration scheme in each case; see FIG. 1):

tapentadol PR 100 mg: 25 mg (bid)->50 mg (bid)->100 mg (bid);

tapentadol PR 200 mg: 100 mg (bid)->150 mg (bid)->200 mg (bid);

oxycodone HCl CR 20 mg: 10 mg (bid)->10 mg (bid)->20 mg (bid).

### Results:

The difference from the adjusted mean square error (±standard error) in the mean pain intensity compared to the placebo was significant for tapentadol PR 200 mg (−8.4 mm [±3.30]; P=0.021). The differences of the adjusted mean square errors (±standard error) in mean pain intensity compared to the placebo was: for tapentadol PR 100 mg−5.9 mm (±3.34; P=0.142) and for oxycodone HCl CR 20 mg−5.4 mm (±3.22; P=0.091), ie tapentadol PR 100 and oxycodone HCl CR 20 mg exhibited similar behaviour (see FIG. 2).

In all the groups, gastrointestinal disorders (included nausea, constipation and vomiting) and disorders of the nervous system (including tiredness and dizziness) were the most common side effects:

| | | Tapentadol | Tapentadol | Oxycodone |
|---|---|---|---|---|
| Side effects | Placebo | PR 100 mg | PR 200 mg | HCl CR 20 mg |
| Gastrointestinal | 23% | 30% | 49% | 56% |
| Constipation | 5% | 7% | 10% | 20% |
| Nervous system | 15% | 24% | 34% | 43% |

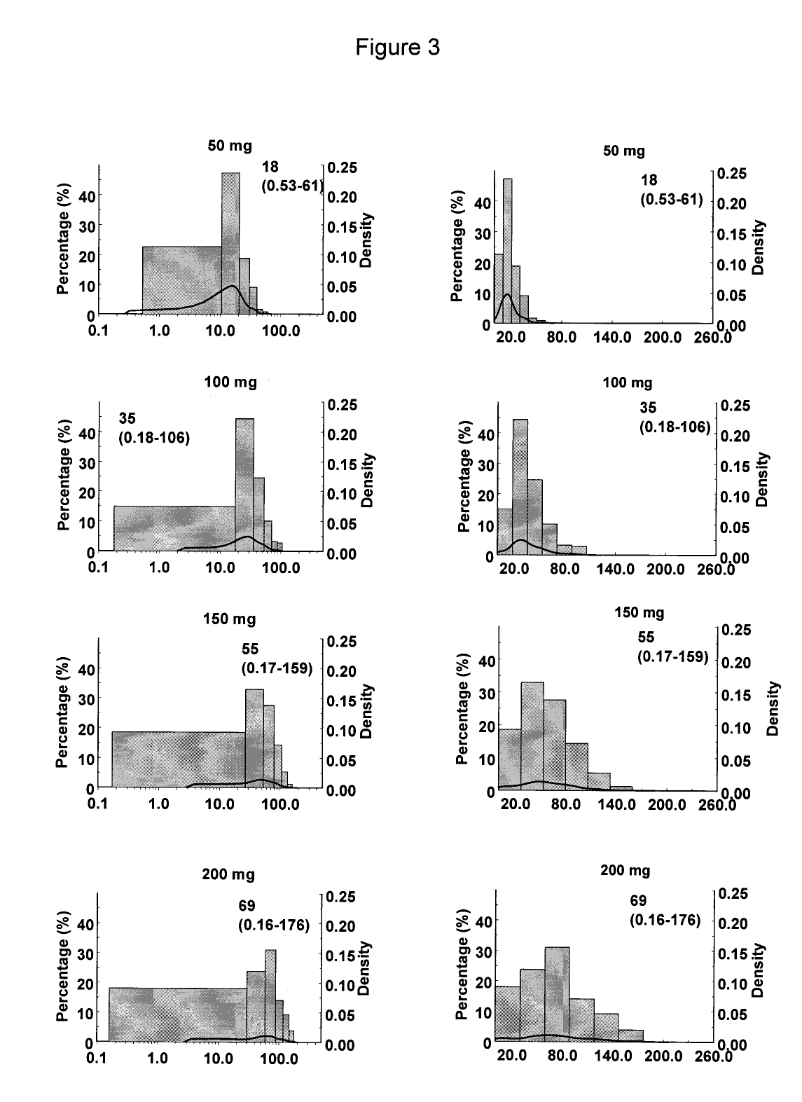

A mathematical evaluation of the distribution of serum concentration within a patient population following the administration different doses of tapentadol is depicted in FIG. 3.

The clinical data confirm that tapentadol PR 200 mg is effective for 4 weeks in the treatment of moderate to severe chronic pain due to arthrosis. With respect to gastrointestinal side effects and side effects associated with the central nervous system, the clinical data indicate that tapentadol is better tolerated than oxycodone.

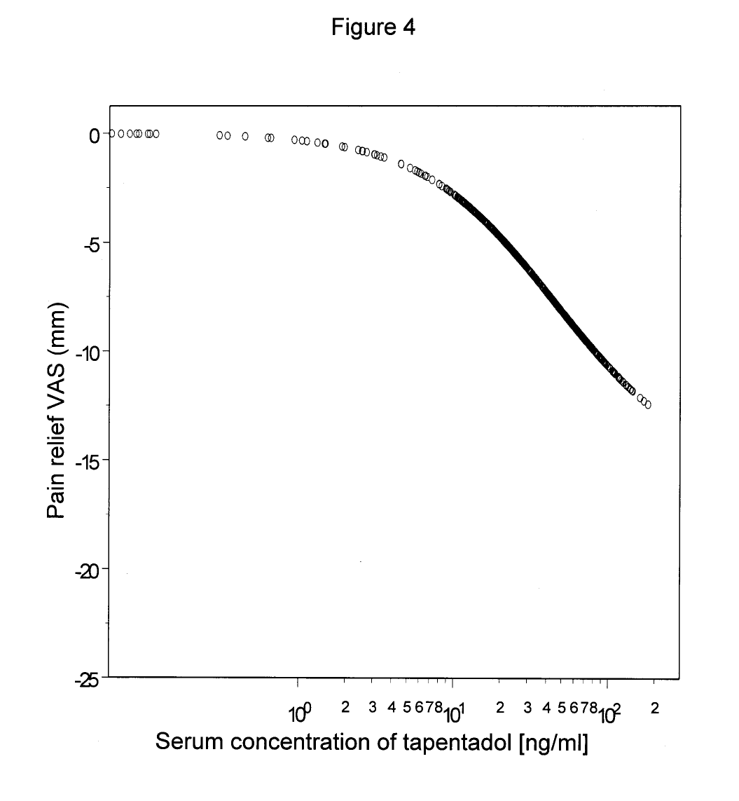

A mathematical evaluation of the connection between serum concentration of tapentadol and efficacy with respect to the alleviation of pain in a patient population based on data from various clinical studies is shown in FIG. 4.

### Example 2

### Methods

### 90-Day Phase III, Double-Blind, Active-Control, Flexible-Dose Study

Patients (N=878) were randomly assigned in a 4:1 ratio to receive tapentadol IR (50 or 100 mg every 4-6 hours as needed; up to 600 mg/day) or oxycodone HCl IR (10 or 15 mg every 4-6 hours as needed; up to 90 mg/day). Pain intensity over the 24 hours prior to each visit was recorded from the date of the first study drug intake through the last visit using an 11-point numerical rating scale (0=“no pain,” 10 “worst possible pain”). Tolerability was assessed starting on the day of the first intake of study drug and concluded 2 days after the final study drug intake.

### Results:

A total of 679 patients in the tapentadol IR group and 170 patients in the oxycodone HCl IR group were included in the efficacy and safety analyses. The pain intensity scores were similar between the groups over time. Mean baseline pain intensity scores were 7.0 for the tapentadol IR group, and 7.2 for the oxycodone HCl IR group. These values decreased at the end of the double-blind period to 4.9 and 5.2 for the tapentadol IR and oxycodone HCl IR groups, respectively. The most common treatment emergent adverse events were nausea, vomiting, dizziness, constipation, headache, and somnolence. Patients in the tapentadol IR group had significantly (P<0.001 for all measures) lower incidences of nausea (18%), vomiting (17%), and constipation (13%), compared with the oxycodone HCl IR group (nausea, 29%; vomiting, 30%; constipation, 27%), while the incidences of somnolence, dizziness, and headache were similar between groups. Serious treatment emergent adverse events were reported by 0.7% and 1.8% of the patients in the tapentadol IR and oxycodone HCl IR groups, respectively. These were not judged by the investigator to be related to the study drug.

### Example 3

### Methods

### Phase III, Double-Blind Study

878. patients were randomly assigned to take tapentadol IR (50 or 100 mg; max, 600 mg/d) or oxycodone HCl IR (active control; 10 or 15 mg; max, 90 mg/d) every 4 to 6 hours as needed for 90 days. Treatment groups were compared with the Cochran-Mantel-Haenszel test.

### Results:

Analyses included 679 and 170 patients in the tapentadol IR and oxycodone HCl IR groups, respectively. Opioid-experienced patients (taking opioids at least 5 days/week for 30 days before screening) made up 49.0% and 48.2% of patients in the tapentadol IR and oxycodone HCl IR groups, respectively. Mean pain scores decreased from baseline to study end for the tapentadol IR (7.0 to 4.9) and oxycodone HCl IR 7.2 to 5.2) groups, respectively. The most common treatment-emergent adverse events (TEAEs) were nausea, vomiting, dizziness, constipation, headache, and somnolence. Significantly (P<0.001) fewer gastrointestinal TEAEs occurred in the tapentadol IR group (nausea, 18%; vomiting, 17%; constipation, 13%) than in the oxycodone HCl IR group (nausea, 29%; vomiting, 30%, constipation, 27%), while the rates of headache, dizziness, and somnolence were similar in both groups. Generally, non-opioid-experienced patients had more TEAEs, but patients taking tapentadol IR were less likely to experience adverse events within this subgroup compared with oxycodone HCl IR patients. For non-experienced patients, vomiting occurred in 18% and 39% in the tapentadol IR and oxycodone HCl IR groups, respectively, while nausea was reported by 22% and 35% of tapentadol IR and oxycodone HCl IR patients, respectively. In opioid-experienced patients, 16% and 21% reported vomiting in the tapentadol IR and oxycodone HCl IR groupd, respectively, while nausea occurred in 14% of patients in the tapentadol IR group and 23% in the osycodone HCl IR group. Prior opioid experience did not reduce the incidence of constipation in either the tapentadol IR (opioid-experience, 17%; non-experienced, 14%) or oxycodone HCl IR (opioid-experienced, 27%; non-experienced, 27% groups.

### Example 4

### Methods

### Phase III, Double-Blind, Placebo- and Active-Controlled, Outpatient Study

674 randomly assigned patients received doses of placebo, tapentadol IR 50 or 75 mg, or oxycodone HCl IR 10 mg every 4 to 6 hours during waking hours. Study endpoints included the sum of pain intensity (SPID) over 5 days (primary endpoint), tolerability assessments, and analyses of age and gender to examine potential differences among subsets of the study population.

### Results:

666 randomly assigned patients were included in the safety analyses; 659 were included in the efficacy analyses. Tapentadol IR 50 and 75 mg showed significant improvements in pain, compared with placebo, bases on 5-day SPID scores (P<0.001). The oxycodone IR 10 mg group also showed significant improvements in 5-day SPID scores (P<0.001) compared with the placebo group, which validated assay sensitivity. Based on pre-specified criteria for 5-day SPID, tapentadol IR 50 and 75 mg were at least as effective as oxycodone HCl IR 10 mg. 5-day SPID scores in all active treatment groups were similar between patients <65 and ≧65 years old as well as between male and female subgroups. Common adverse events included gastrointestinal (GI) and central nervous system disorders. Overall, the incidence of GI adverse events showed a dose response relationship for the tapentadol IR 50 and 75 mg groups (29% and 40%, respectively) that was lower than oxycodone HCl IR 10 mg (69%). These trends were also evident within the subgroups. Patients <65 and ≧65 reported fewer GI adverse events for tapentadol IR 50 mg (25% and 36%, respectively) than 75 mg (42% and 38%, respectively), and both were less than oxycodone HCl IR 10 mg (66% and 74%, respectively). The incidence of GI adverse events reported by the male and female subgroups were 21% and 39%, respectively for tapentadol IR 50 mg, 28% and 54%, respectively for tapentadol IR 75 mg, compared with 58% and 81%, respectively for oxycodone HCl IR 10 mg.

### CONCLUSIONS

The clinical data demonstrate the efficacy of tapentadol IR for the treatment of chronical pain due to osteoarthritis. As regards gastrointestinal adverse events the clinical data shows an improved tolerance of tapentadol compared to oxycodon HCl.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. A method of treating pain due to arthrosis in a subject in need thereof, said method comprising administering to said subject a pharmacologically effective amount of tapentadol.

2. A method according to claim 1, wherein the mean serum concentration of tapentadol following twice-daily administration over a period of at least three days is on average at least 5.0 ng/ml.

3. A method according to claim 1, wherein the mean serum concentration of tapentadol in at the most 50% of the patient population following twice-daily administration over a period of at least three days is on average less than 5.0 ng/ml.

4. A method according to claim 1, wherein the mean serum concentration of tapentadol in at the most 50% of the patient population, following twice-daily administration over a period of at least three days is on average more than 300 ng/ml.

5. A method according to claim 1, wherein the mean serum concentration of tapentadol in at least 50% of the patient population, following twice-daily administration over a period of at least three days is on average in the range of from 1.0 ng/ml to 500 ng/ml.

6. A method according to claim 1, wherein the tapentadol is administered in a solid dosage form.

7. A method according to claim 1, wherein the tapentadol is administered orally.

8. A method according to claim 1, wherein the tapentadol is administered twice-daily.

9. A method according to claim 1, wherein the tapentadol is administered in an amount of 10 to 300 mg.

10. A method according to claim 1, wherein the tapentadol is administered in a pharmaceutical composition further comprising a pharmaceutically acceptable carrier.

11. A method according to claim 1, wherein the tapentadol is administered in pharmaceutical dosage form having a total mass in the range of from 25 to 2,000 mg.

12. A method according to claim 6, wherein said solid dosage form is selected from the group consisting of tablets, capsules, pellets and granules.

13. A method according to claim 1, wherein the arthrosis is selected from the group consisting of gonarthrosis, coxarthrosis and spondylarthrosis.

14. A method according to claim 1, wherein said pain is moderate to strong pain.

15. A method according to claim 1, wherein said pain is selected from the group consisting of pain following periods of inactivity, weight-bearing pain, fatigue-induced pain, periarticular pain on pressure, radiating pain, pain at rest after spending a long time in the same position, constant pain, spontaneous pain, pain on movement, night pain, muscular pain, pain at the end of the range of movement and osseous pain as spontaneous pain and pain at rest.
